(19) United States
(12) Patent Application Publication
(10) Pub. No.: US 2011/0144739 A1
(43) Pub. Date: Jun. 16, 2011

(54) MEDICAL DEVICE AND METHOD FOR PRODUCING A DEVICE OF SAID KIND

(75) Inventor: Giorgio Cattaneo, Karlsruhe (DE)

(73) Assignee: ACANDIS GMBH & CO. KG, Pfinztal (DE)

(21) Appl. No.: 13/057,568

(22) PCT Filed: Aug. 3, 2009
(86) PCT No.: PCT/EP2009/005606

§ 371 Date: Feb. 4, 2011

(30) Foreign Application Priority Data
Aug. 5, 2008 (DE) 10 2008 036 428.2
Sep. 23, 2008 (DE) 10 2008 048 416.4

(51) Int. Cl. A61F 2/82; B21D 11/14

(52) U.S. Cl. 623/1.22; 72/371

(57) ABSTRACT

A medical device is provided with a rotation-symmetrical lattice structure (10) having at least two wire elements (11, 12) which are wound about a common rotational axis R in a spiral shape and form first and second intersection points S′, S″ with a common plane L arranged perpendicular to the rotational axis, wherein a first straight line R′ runs through the first intersection point S′ and a second straight line R″ runs through the second intersection point S″, the straight lines being arranged parallel to the rotational axis and enclosing an acute angle (α′, α″) with one of the two wire elements (11, 12). The two angles (α′, α″) are different from each other. A method for producing a device of this kind is also provided.

The invention relates to a medical device according to the preamble to claim 1 and a method for producing a device of said kind according to the preamble to claim 22. A medical device of the type described in the introduction is known, for example, from U.S. Pat. No. 6,258,115 B1.

U.S. Pat. No. 6,258,115 B1 discloses a stent with a rotation-symmetrical tubular lattice structure formed from a wire braid. The mesh size of the wire braid is partially different so that the stent has different degrees of permeability. Preferably, a middle portion of the stent has a larger mesh size so that, when used in the region of a vascular bifurcation, the stent allows a sufficient blood flow into a collateral vessel.

The porosity or permeability of the stent portions is determined inter alia by the braiding angle. The braiding angle is defined as the angle formed between a wire wound in a spiral shape about the rotational axis of the stent in top view with a straight line parallel to the rotational axis or a projection of the rotational axis into the circumferential plane. When comparing the braiding angles of two wires of the stents, wherein the angles to be compared are observed in the same cross-sectional plane of the stent, it should be established that the two braiding angles are the same. A variation of the braiding angle to change the permeability properties of the stent is only performed in the axial direction of the stent. This is in particular evident when observing the intersection points or knotted points at which two wires running about the rotational axis intersect. The two wires have the same angle relative to a straight line parallel to the rotational axis straight line running through the intersection region.

Generally, devices with a rotation-symmetrical lattice structure comprising two wire elements wound in a spiral shape about a common rotational axis, i.e. braids or coils, are used in medical technology to reinforce supply systems, for example catheters or tubes. Supply systems of this kind are used inter alia to supply substances, medicines or contrast media into or out of the body or for diagnosis, in particular for measuring temperature or pressure. A further possible application of braids or coils of this kind relates to the reinforcement of tubes for endoscopy. Hereby, the wire framework incorporated in tubes, which are generally made of plastic, serves to stabilise the tube. In particular, this should prevent the tube from buckling in narrow bending radii.

Known wire braids or wire spirals have the property that the cross-sectional diameter of the rotation-symmetrical lattice structure changes in dependence on a change in the length of the wire braid. For example, shortening the braid or coil results in the widening of the cross-sectional diameter, while a lengthening the braid or coil effects a reduction of the cross-sectional diameter. In the case of plastic-sheathed wire structures with small wall thicknesses, for example in the case of a catheter reinforced with a wire braid, pushing the catheter in the blood vessel results in a shortening of the catheter length, which increases the cross-sectional diameter of the catheter. As a result, the catheter comes into contact with the vessel wall, i.e. the friction between the catheter and vessel wall is increased so that further advancement of the catheter is prevented or at least hampered. Here, there is a risk of the widened catheter closing the vessel or reducing the blood flow, which could be associated with implications for the patient's health.

The reverse applies when the catheter is pulled, in particular the wire braid or coil inside the catheter. The pulling effects a lengthening of the catheter, which reduces the cross-sectional diameter of the tube. In the case of aspiration catheters, a reduction of the inside width causes, for example, a loss of suction pressure. In the case of catheters used to introduce implants into the human body, a radial pressure is applied to the implant carried in the catheter and this can result in damage to the implant.

In principle, in particular with long catheter systems, the described length change makes it more difficult to position the catheter tip on the desired target or treatment site. Hereby, the wire braid or the wires wound in a spiral shape have a spring-like action so that the pushing or pulling of the catheter by the user, i.e. outside the body, is not implemented identically intracorporeally at the catheter tip.

Although the wire structure is at least partially reinforced by embedding in a plastic or enclosing in a plastic if the wall thickness of the enclosure is suitably high, at the same time, however, this reduces the flexibility of the system, since, due to the relatively high wall thickness, the plastic can only expand or contract poorly. In particular, with small bending radii, despite the stabilising effect of the wire braid, there is a risk of the plastic layer buckling, which impairs the functionality of the system, in particular in the case of catheters.

One special application for known wire-reinforced tubes is the stabiliser, which is used to push implants out of a catheter into a vessel. Hereby, the stabiliser, i.e. a wire-reinforced tube, is located inside the catheter tube. Catheter systems of this kind are preferably relatively small so they can be used in small vessels, for example coronary vessels or intracranial vessels. With small dimensions and tolerances of this kind, an increase in diameter caused by the pushing of the stabiliser results in a blockage of the system so that, due to the increased friction between the inner wall of the outer catheter tube and the actual stabiliser, the stabiliser cannot be pushed any further in the direction of the treatment site. To prevent the diameter changing when the stabiliser is pushed, it is possible to reinforce the wire braid additionally by increasing the wall thickness of the plastic coating. This increases the overall diameter of the catheter system, which precludes treatment of small vessels.

The invention is therefore based on the object of providing a possibility for influencing the radial stability of a medical device.

According to the invention, this object is achieved with respect to the medical device by the subject matter of claim 1 and with respect to the method for producing a device of said kind by the subject matter of claim 22.

The invention is based on the concept of disclosing a medical device comprising a rotation-symmetrical lattice structure with at least two wire elements wound in a spiral shape about a common rotational axis R, which form first and second intersection points S′, S″ with a common plane arranged perpendicular to the rotational axis R, wherein a first straight line R′ runs through the first intersection point S′ and a second straight line R″ runs through the second intersection point S″, said straight lines being arranged parallel to the rotational axis R and enclosing an acute angle with one of the two wire elements. Hereby, the two angles are different.

The configuration of the braiding angles, i.e. the angles α′ and α″, relates to the idle state of the device. The determination of the braiding angle is performed with the device in relaxed state, i.e. without exposure to external forces. Preferably, the device is aligned in a straight line in the axial direction for the determination of the angle.

Hence, the invention relates to a new type of braid configuration or alignment of the wire elements for medical devices, wherein the angle between a first wire element and a perpendicular projection of the rotational axis R onto the circumferential plane of the lattice structure is different from the angle between a second wire element and a further perpendicular projection of the rotational axis R onto the circumferential surface of the lattice structure. The comparison of the angles is performed at the same axial height of the lattice structure, i.e. in a cross-sectional plane L of the lattice structure oriented perpendicular to the rotational axis R. In this way, a lattice structure is provided which is asymmetrical with respect to the angles or braiding angles. If there is a change in the diameter or length, separate wire spirals, with different angles, behave differently. In the case of a combination of the wire elements with different angles wound in a spiral shape to form the lattice structure, this difference causes the wire elements to block each other in their freedom of movement so that a change in diameter is prevented. In this way, a dimensionally or inherently stable structure is provided.

When the medical device according to the invention is used for the reinforcement of medical tubes, for example catheters, buckling or collapsing is impeded, since the stabilisation of the tube by the asymmetrical braid prevents a change in diameter. For the purposes of the invention, an acute angle is an angle greater than 0° and smaller than 90°.

In a preferred embodiment of the medical device according to the invention, all the wire elements of the lattice structure comprise the same inelastic material so that the lattice structure substantially has a rigid, invariable or stable geometry. In this way, the diameter of the lattice structure, or its shape generally, is not only defined by the braid configuration or the geometry, but also by the properties of the material. The rigid structure, and the stability of length diameter associated therewith, ensures that, for example, a catheter reinforced by means of the medical device or a catheter embodied as the medical device can be exactly positioned.

In an alternative embodiment, all the wire elements of the lattice structure comprise the same elastic material so that the rotation-symmetrical lattice structure has a substantially variable geometry. The elastic material achieves the possibility of a selective change in the diameter and length of the medical device. This is interesting, for example, when the medical device is used as stent, since in this way it is possible to set or reinforce a restoring force in the radial direction by means of which the medical device or the stent in implanted state can be pressed radially against the vessel wall and hence anchored. Furthermore, the elastic wire elements enable the medical device to follow a movement of the vessel wall, for example due to the pulse beat.

Preferably, the angle difference between the first angle and the second angle is at least 2°, in particular at least 5°, in particular at least 8°, in particular at least 10°, in particular at least 20°, in particular at least 30°, in particular at least 40°, in particular at least 45°, in particular at least 50°, in particular at least 60°, in particular at least 70°, in particular at least 90°. Generally, the radial stability of the medical device is determined by the size of the difference between the first and the second angle or by the degree of asymmetry. The greater the difference between the angles, the greater the degree by which the asymmetrically running mutually associated wires or wire elements block each other.

Furthermore, the angle difference between the first angle and the second angle can be at most 50°, in particular at most 45°, in particular at most 40°, in particular at most 30°, in particular at most 20°, in particular at most 10°, in particular at most 8°, in particular at most 6°, in particular at most 4°, in particular at most 2°. The force required to change the shape or extend the length of the wire elements increases as the difference between angles increases. In conjunction with the angle difference, a suitable choice of the modulus of elasticity of the wire elements enables the inherent stability or a radial restoring force to be selectively set. The higher the modulus of elasticity, that is the weaker the extensibility of the wire elements and/or the greater the angle difference, the higher the inherent stability of the braided mesh structure. The smaller the angle difference, the more extensible a tubular lattice structure, since the wire elements wound in a spiral shape with a flat angle permit a higher axial length variation. The upper limit of the angle difference of 10°, in particular of 8°, in particular of 6°, in particular of 4°, in particular of 2°, is particular suitable for medical purposes, since, with relatively low external forces, for example in the case of crimping or when implanted in the vessel, a longitudinal extension of the wires and hence a restoring force is effected. Angle differences of less than 2° are possible.

If the emphasis is on the establishment of a restoring force that reinforces the radial force, for example in the case of a stent or another implant, a certain extensibility or longitudinal deformability of the wire elements is required. The wire elements are elastic. Suitable materials are disclosed in the application. Due to the asymmetrical wire elements with mutual blocking of their freedom of movement, due to exposure to an external force, a length variation of at least the wire elements with a flatter braiding angle is achieved, and as a result, they exert a restoring force on the braid, which intensifies the radial force. For this, a braiding angle difference within the range of 2° to 10° is advantageous, since a relatively low external force suitable for medical purposes is sufficient to create the restoring force.

The first angle can be smaller than 45°, in particular smaller than 40°, in particular smaller than 20°.

The second angle can be greater than 2°, in particular greater than 3°, in particular greater than 5°, in particular greater than 7°, in particular greater than 45°, in particular greater than 50°, in particular greater than 70°.

Furthermore, the ratio of the number of the first wire elements, i.e. the wire elements with the first angle, and the number of the second wire elements, i.e. the wire elements with the second angle, can be at most 1:1, in particular at most 1:2, in particular at most 1:4, in particular at most 1:6, in particular at most 1:8, in particular at most 1:12, in particular at most 1:24. Preferably, the first wire elements have a smaller angle than the second wire elements. Due to the asymmetrical geometry, the first wire elements, or generally wire elements with a relatively small angle, are more greatly extended than wire elements arranged in a greater angle to the rotational axis R and as a result create a restoring force so that, in the case of wire structures with a low radial force, the force is increased. The fact that the number of the first wire elements with a smaller angle is lower than the number of the second wire elements enables the restoring force of the lattice structure to be finely set.

In principle, an extension of the wire elements with small angle differences between the first and second wire elements is possible. Unlike the case with symmetrical braid configurations, tubular mesh structures for example, in particular stents, have a higher radial force due to the asymmetrical arrangement of the wire elements, since the wire elements with a smaller angle form wire spirals which are pulled apart or extended in the axial direction with a lower force. If the difference between the first and the second angles α′, α″ is low, in particular in the range of from 2° to 10°, the external forces required for the extension of the wire elements are so low that a lattice structure designed in such a way is suitable for medical applications. The external forces effecting the extension form, for example, during the transferral of a tubular lattice structure or a stent from an expanded state into a compression state, i.e. during the crimping of the stent. In implanted state, the stent is also at least partially compressed.

In a preferred embodiment of the medical device according to the invention, at least one stabilisation section is provided which extends at least partially in the axial direction along the lattice structure, wherein the first angle along the stabilisation section is always greater or always smaller than the second angle is. In this way, it is possible to achieve a widening or compression of the lattice structure in some areas, namely in the sections arranged outside the stabilisation section, while the stabilisation section has a constant, invariable diameter.

The stabilisation section can form an axial central section and/or an axial end section of the lattice structure. The formation of an axial central section as a stabilisation section enables the device to be used, for example, as an implant, in particular as a stent, in a vessel, wherein the stabilisation section prevents the collapse of the vessel or increases the radial force for the widening of the vessel, since in the stabilisation section, a change in diameter is prevented or the radial force is increased, while, on the other hand, due to a possible change in diameter in the end regions, the medical device can follow the movement of the vessel wall, for example in the case of pulsation (compliance). This enables the provision of stable and easy-to-fix artificial vessels or vascular prostheses. Furthermore, medical devices can have a first axial end section with an asymmetrical braid and a second axial end section comprising a symmetrical or flexible braid configuration. The first, stabilised end section permits the simple pushing of the device, while, on the other hand, the second, flexible end section can expand and fulfil a correspondingly desired function.

The medical device can comprise two stabilisation sections each forming an axial end section of the lattice structure. The arrangement of the stabilisation section in the two end sections of the lattice structure has the advantage that the end sections have a stabilising function and a central section has, for example, a braid configuration permitting radial expansion or compression. The stabilisation section can extend over the entire lattice structure.

Particularly preferred is an embodiment of the medical device comprising two stabilisation sections each forming an axial end section of the lattice structure. For example, this enables particularly stable and simple-to-fix artificial vessels or vascular prostheses.

Preferably, the first angle and/or the second angle varies at least in sections along the lattice structure, in particular along a straight line R′, R″ running parallel to the rotational axis R. This design is advantageous, since the properties of the lattice structure are variable, in particular continuously variable or adjustable along the rotational axis R.

In a preferred embodiment of the medical device according to the invention, the rotation-symmetrical lattice structure is embodied substantially as a tube, in particular as a stent. In this way, the device is particularly suitable for the reinforcement or stabilisation of tubes with a constant diameter. Furthermore, the tubular lattice structure enables the device to be used as an implant for supporting body vessels, in particular blood vessels.

Preferably, the wire elements are wound in a coil shape along the lattice structure at least in sections. The coil-shaped or spiral-shaped arrangement of the wire elements enables the device according to the invention to be produced simply.

The wire elements can be braided with each other along the lattice structure at least in sections. With a braided structure, in each case one wire element is guided over and under further wire elements so that the frictional force between the wire elements further increases the stability and rigidity of the lattice structure.

Furthermore, the wire elements can be connected to each other at least at an axial end or in an intersection region of the lattice structure, in particular by a positive, non-positive and/or force-fit connection, in particular glued or welded. For example, the wire elements can be connected to each other at an axial end of the lattice structure in such a way that the lattice structure is substantially formed from a coherent wire, which is diverted at each of the axial ends and guided in the reverse direction. In this way, open wire ends at the axial ends of the lattice structure are avoided and, for example, when the device is used as a stent, the risk of injury reduced. Furthermore, the interconnection of the wire elements, in particular in an intersection region, enables a further, improved stabilisation of the lattice structure, since the intersecting wire elements are prevented from sliding on each other. Preferably, the wire elements are connected at the axial ends of the stabilisation section or the lattice structure so that the blocking of the wire elements is ensured.

Preferably, the intersection region in which the wire elements are connected to each other abuts the stabilisation section. This prevents a relative movement of the wire elements among themselves, which is possible in sections of the lattice structure which do not form a stabilisation area, being transmitted into the stabilisation section, in particular the section with the asymmetrical braid configuration. The intersection regions in which the wire elements are connected to each other represent a demarcation of the stabilisation section and facilitate a simple-to-define separation, established during production, between the stabilisation section and further sections of the lattice structure. It is also possible that, in the region of the asymmetrical braid, the geometry, or the frictionally engaged connection in the intersection regions, prevents a relative movement of the wire elements toward each other, without the intersection regions or points of intersection also being fixed, since the braid generally has an inherent stability.

In a preferred embodiment of the medical device according to the invention, the wire elements comprise a shape-memory material and/or a pseudoelastic material, in particular a nickel-titanium alloy. Materials of this kind have high resistance and biocompatibility and also enable a definable change in diameter or radially acting for components of the lattice structure to be adjustable within the scope of the pseudoelastic properties.

Alternatively, the wire elements can comprise a plastic, in particular polyester, polyamide, polypropylene or polyethylene, in particular HDPE or UHMWPE. In this way, the lattice structure can be provided with an elastic variable geometry, which is achieved by the elastic properties of the plastic used. In particular, a suitable choice of the plastic enable deformability and change in diameter or the radial force component of the lattice structure to be set.

In a preferred embodiment, a flexible enclosure is provided, which extends at least in sections in the circumferential direction and/or axial direction along the lattice structure. The enclosure can be arranged on an external or internal circumference of the lattice structure or enclose the wire elements completely in such a way that the lattice structure, at least in sections, is completely embedded in the enclosure. Due to the combination of the asymmetrically braided lattice structure with a flexible enclosure, in particular with tubular mesh structures, tube-like configurations are provided having particularly high stability and strength in the radial direction, wherein the tubes furthermore have sufficient flexibility to be bent or buckled in the axial direction. In this way, it is possible, for example, to provide catheter tubes, which in every operating mode, have a substantially uniform diameter and are simultaneously sufficiently flexible to be manoeuvred by bending of body vessels. The flexible enclosure preferably comprises a plastic, in particular polyurethane, silicone or Teflon.

In a further preferred embodiment of the invention, a plurality of first wire elements each have the first angle α′ (first braiding angle) and together form a first symmetrical structure. A plurality of second wire elements have the second angle α″ (second braiding angle) and together form a second symmetrical structure. The first structure and the second structure are superimposed in such a way that the first and second wire elements are associated with each other with different first and second angles α′, α″. Therefore, with this embodiment, two different structures are provided which are each embodied as inherently symmetrical, that is each have the same braiding angle within the structure. The two structures are combined with each other and interact by means of friction forces, wherein the wire elements of the different structures, that is the first and second wire elements have different braiding angles. The superimposition of the first and second structures overall creates an asymmetrical lattice structure comprising two different substructures which are inherently symmetrical and different with respect to the braiding angle.

It is also possible for more than two structures to be provided, which are inherently symmetrical and, combined with each other, overall produce an asymmetrical overall structure, wherein the overall structure is different, i.e. has three or more different braiding angles. The structures are superimposed and connected to each other or interact in such a way that the transmission of force between the wire elements or the substructures is possible. The substructures are preferably braided with each other or woven. Preferably, the substructures are connected in the intersection regions and/or at the axial ends the lattice structure and/or at the edges of a stabilisation section.

For the asymmetry of the overall braid, it is sufficient for first and second symmetrical substructures with different braiding angles to interact. Further inherently symmetrical third, fourth or more substructures, which are either superimposed or in arranged in sequence in the longitudinal direction, can have the same braiding angle as the first or second substructure or a braiding angle which is different from the braiding angles of the first and second substructure.

The advantage of the device formed from different substructures consists in the fact that the device has particularly high stability against torsion forces.

With respect to the production method for the medical device according to claim 1, the invention is based on the concept of [w]inding at least two wire elements in a spiral shape about a common rotational axis, in such a way that the wire elements each enclose different acute angles with a straight line R′, R″ running parallel to the rotational axis R in at least one plane L arranged perpendicular to the rotational axis R. Particularly preferred is the braiding or winding of the wire elements with a textile machine, in particular a spinning machine or weaving machine.

The invention is described below in more detail using exemplary embodiments with reference to the attached schematic drawings, which show:

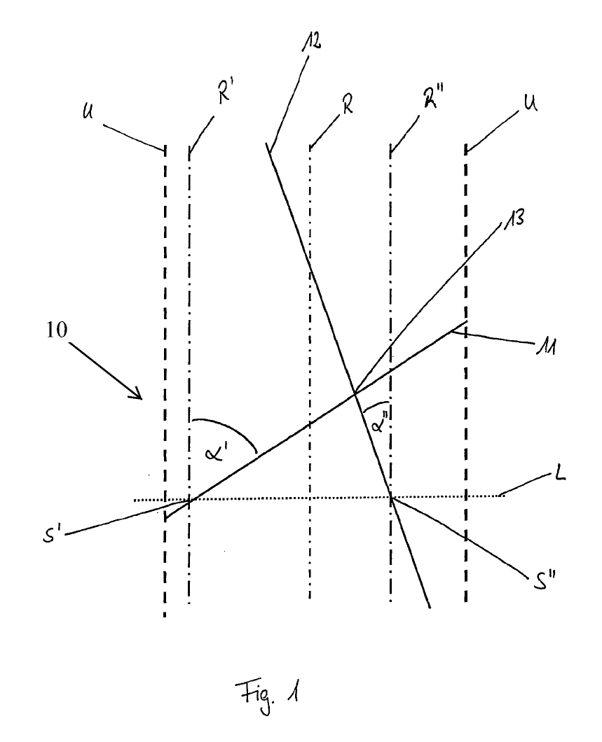

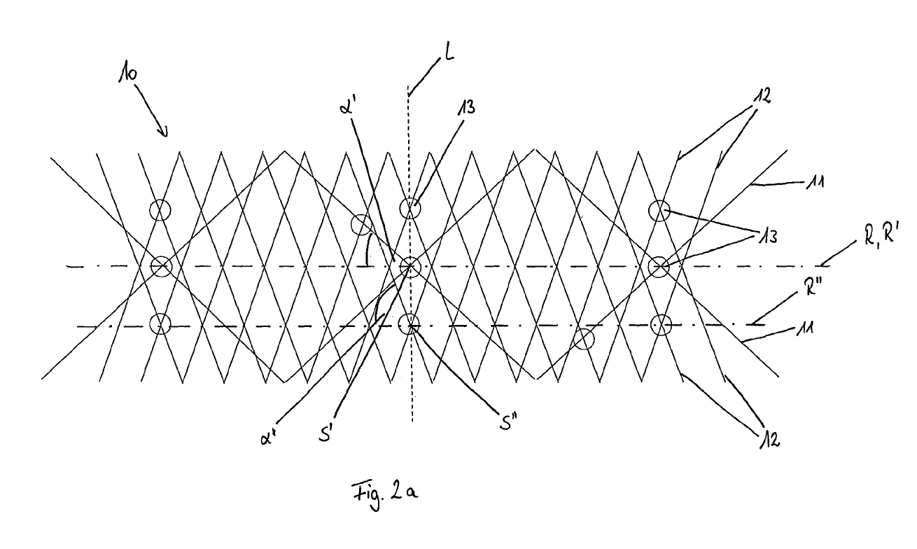

FIG. 1 the arrangement of two wire elements of the medical device according to a preferred exemplary embodiment;

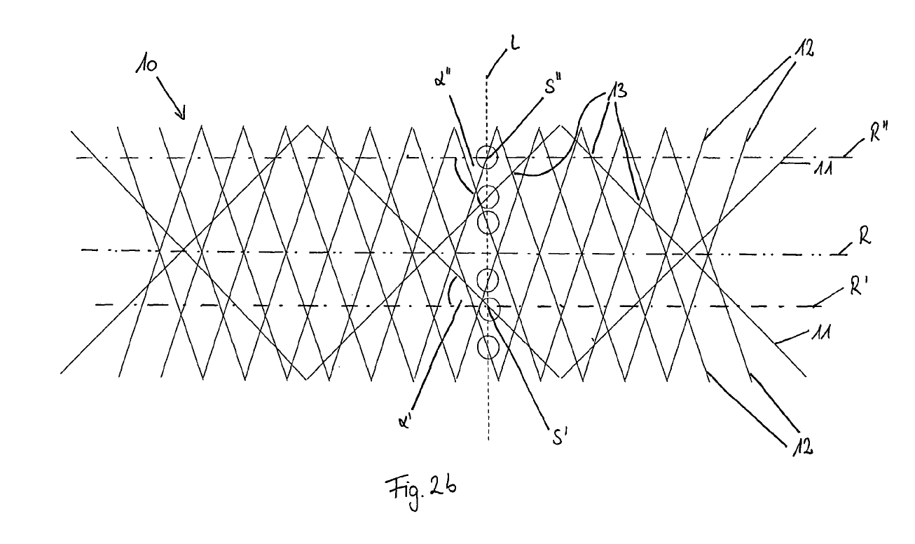

FIG. 2a, 2b a braid configuration of a medical device according to the invention according to a further exemplary embodiment

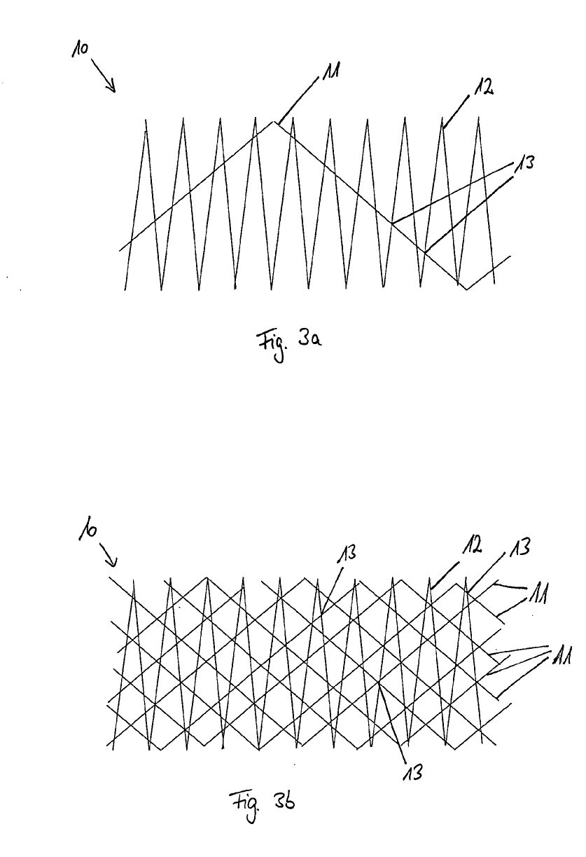

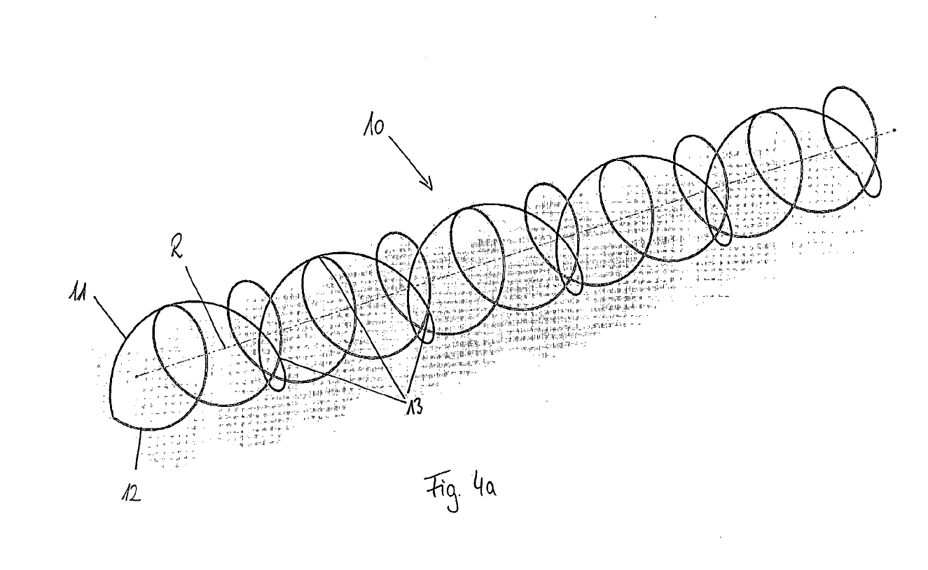

FIG. 3a, 3b a further braid configuration of the medical device according to the invention according to a further exemplary embodiment

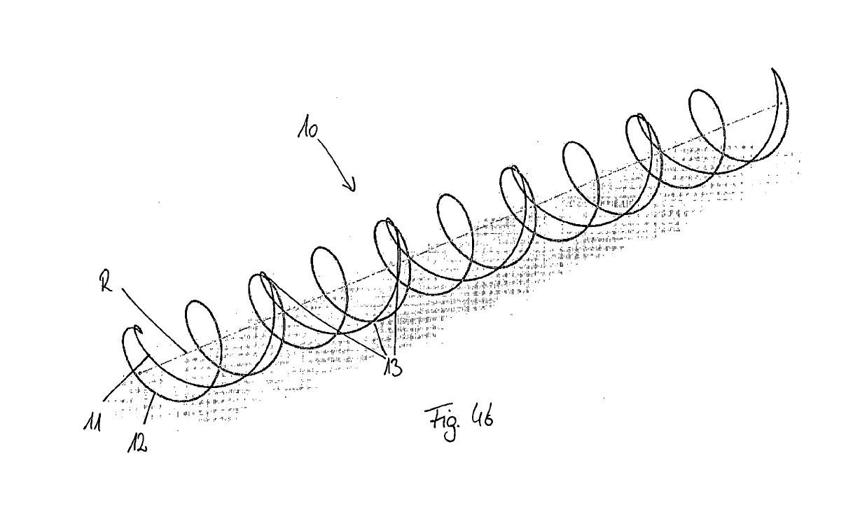

FIG. 4a, 4b a perspective view of medical device according to the invention according to a further, preferred exemplary embodiment, and

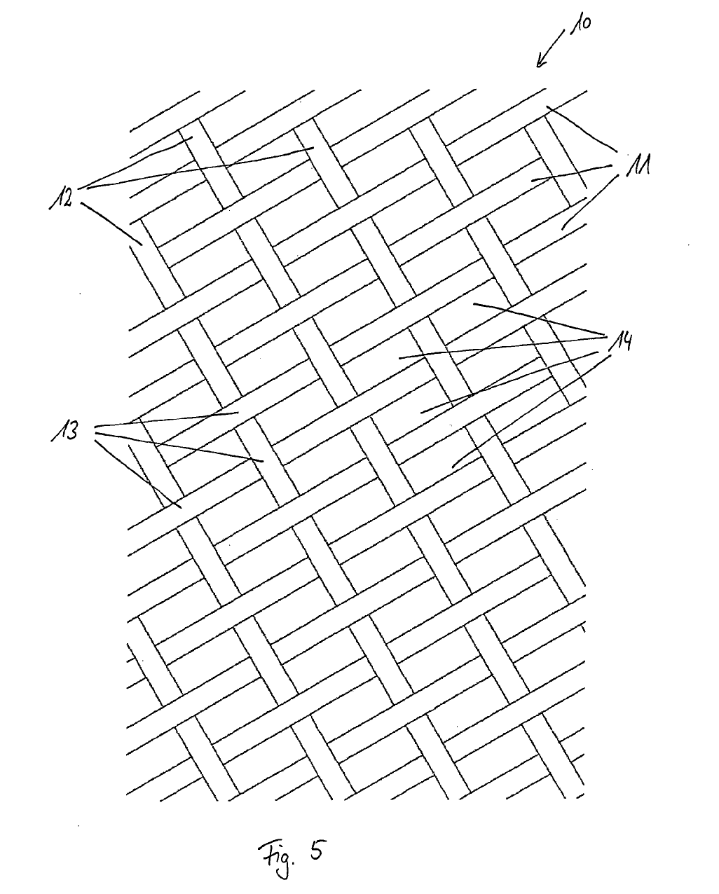

FIG. 5 a detailed view of a braid configuration for a medical device according to the invention according to a further exemplary embodiment.

In FIG. 1 shows by way of example the arrangement of two wire elements 11, 12, wherein in particular the straight line R′, R″, the plane L and the intersecting points S′, S″ formed by the straight line R′, R″ and the plane L are shown. The plane L and the straight line R′, R″ are imaginary reference lines or an imaginary reference surface for the determination of the braiding angle. For reasons of clarity, FIG. 1 only shows two wire elements 11, 12. In principle, the lattice structure 10 can comprise a plurality of wire elements 11, 12. In the representation according to FIG. 1, the lattice structure 10 is shown in unfolded state, i.e. the distance between the demarcation lines U corresponds to the circumference of the lattice structure 10. The wire elements 11, 12 overlap and thereby form an intersection region 13. The tubular lattice structure according to FIG. 1 is embodied as rotation-symmetrical with respect to a rotational axis R. A cross-sectional plane L arranged perpendicular to the rotational axis R forms an intersection point S′, S″ with each of the wire elements 11, 12. A straight line R′, R″ arranged parallel to the rotational axis R and to the circumferential surface of the lattice structure 10 runs through each of the intersecting points S′, S″. Hereby, the angle α′ formed between the first wire element 11 and the first straight line R′ in the region of the first Intersecting point S′ corresponds to the braiding angle of the first wire element 11. The same applies to the second angle α″ formed in the region of the second intersecting point S″ by the angle between the second wire element 12 and the second straight line R″. The second angle α″ corresponds to the braiding angle of the second wire element 12. In principle, the acute angles, i.e. angles with a value greater than 0° and smaller than 90°, are used for the comparison of the angles α′, α″ of the two wire elements 11, 12. It is also possible to use the obtuse angle. It is also possible for the corresponding angles to be compared, i.e. the acute angles or the obtuse angles are compared with each other.

As can be seen in FIG. 1, the two angles α′, α″ have different sizes. To be specific, the angle α′ is greater than the angle α″. Consequently, the wire element 12 with the angle α″ has a greater inclination than the wire element 11 with the angle α′, wherein the inclination corresponds to an imaginary axial displacement along the longitudinal axis in the case of a full revolution.

The wire elements 11, 12 are generally wound in a spiral shape about the rotational axis R, wherein the wire elements 11, 12 can be arranged in same direction or in opposite directions along the rotational axis R. The individual wire element 11, 12 consequently has a substantially spring-shaped structure, wherein the inclination of the wire element 11 wound in a spiral shape differs from that of the wire element 12 due to the different angles α′, α″. The wire elements 11, 12 themselves could have round or angular cross sections. At least at the ends of the lattice structure 10, the wire elements 11, 12, in particular the free wire ends 11′, 12′, can be connected to each other, for example by means of a positive connection. For example, the wire elements 11, 12 can be welded or glued to each other. The wire elements 11, 12 described here form the lattice structure 10, i.e. the wire elements 11, 12 are arranged in such a way in a spiral shape on the circumference of the lattice structure that the cross-sectional diameter of the spiral formed corresponds to the cross-sectional diameter of the lattice structure 10. Consequently, the lumen of the medical device is defined by the cross-sectional diameter of the individual wire spirals 11, 12.

The invention is not restricted to strictly rotation-symmetrical configurations. Instead, the invention covers implants or medical devices such as catheters or stabilisers, whose walls form an inner lumen or an inner compartment, wherein the walls exert an outwardly-directed radial force on the vessel wall coming into contact therewith. This is, for example, also possible with a body having a hollow-oval or other cross section. Furthermore, it is sufficient for the implant or the medical device to be embodied as rotation-symmetrical or approximately rotation-symmetrical at least in sections, where further sections of the implant or the medical device can be embodied as non-rotation-symmetric.

FIG. 2a shows a further exemplary embodiment of the medical device according to the invention comprising a plurality of wire elements 11, 12 forming the lattice structure 10. Hereby, the lattice structure 10 is again shown in unfolded state so that the entire circumferential surface of the lattice structure 10 is depicted in the plane of the drawing. The lattice structure 10 comprises two first wire elements 11, which superimpose a total of four second wire elements 12. A different number of first and second wire elements 11, 12 is possible. Hereby, the first wire elements 11 comprise a smaller braiding angle α′ than the second wire elements 12, wherein the rotational axis R of the lattice structure 10 according to FIG. 2a is arranged horizontally in the plane of the drawing. Furthermore, FIG. 2a highlights, by means of circular markings, a plurality of intersection regions 13 in which a first wire element 11 intersects a second wire element 12. It is also possible for a of plurality wire elements 11, 12 to intersect in an intersection region 13. Also shown is a plane L running through three intersection regions 13 in each of which two first wire elements 11 or two second wire elements 12 intersect. In this case, the intersecting points S′, S″ between the plane L and the wire elements 11, 12 are arranged in the intersection regions 13. Hereby, the intersection point S′ between the two first wire elements 11 and the plane L lies on the straight line R′, which, in the representation according to FIG. 2a, is identical to the rotational axis R or superimposes the rotational axis R. The second straight line R″ is arranged parallel to the rotational axis R and runs through the second intersection point S″ formed by the plane L and at least one, here two, second wire elements 12. In the event of the plane L running through an intersection region 13 of a first wire element 11 and a second wire element 12, the first intersection point S′ and the second intersection point S″ and the two straight line R′, R″ coincide or are identical. The angles α′, α″ formed between the straight line R′, R″ and the respective associated wire elements 11, 12 have different values. In particular, the second wire elements 12 are arranged under a greater braiding angle than the first wire elements 11, i.e. the wire spirals formed by the first wire elements 11 have a greater inclination than the wire spirals formed by the second wire elements 12. In principle, the wire elements 11, 12 are arranged on the same circumference of the lattice structure 10, i.e. the spiral rotation bodies formed by the wire elements 11, 12 comprise substantially the same cross-sectional diameter. Hereby, the wire elements 11, 12 can be braided or woven with each other or superimpose each other. For example, a first wire element 11 is arranged on the outer circumference of a second wire element 12 wound in a spiral shape, wherein the first wire element 11 has the same rotational axis R as the second wire element 12.

FIG. 2b shows substantially the same lattice structure 10 as FIG. 2a, wherein the viewing plane L is arranged offset in the axial direction compared to the representation according to FIG. 2a. Hereby, the viewing plane L is arranged in such a way that the plane L does not run through any intersection region 13. The intersecting points S′, S″ are, therefore, each formed from only one wire element 11, 12 and the plane L. In FIG. 2b, the intersecting points S′, S″ are highlighted by circular marks. The angles α′, α″ between the wire elements 11, 12 and the straight lines R′, R″ arranged parallel to the rotational axis R are also different. It is evident from FIGS. 2a and 2b that the different angles α′, α″ are independent of the position of the viewing plane L. However, the arrangement of the reference lines R′, R″, i.e. the observation of the acute angle between the wire elements 11, 12 and the respective projection of the rotational axis R onto the circumferential plane in the intersection point S′, S″, is relevant for the determination the angle α′, α″.

FIG. 3a shows a further exemplary embodiment of the medical device according to the invention, wherein the lattice structure 10 is formed from two wire elements 11, 12 with different angles α′, α″. Hereby, the first wire element 11 superimposes the second wire element 12. In the intersection regions 13, the wire elements 11, 12 are in contact, wherein the first wire element 11 can run both completely outside the second wire element 12 and completely below the second wire element 12. It is also possible for the first wire element 11 to intersect the second wire element 12 partially above and partially below it, in particular in such a way that the first wire element 11 is braided with the second wire element 12.

FIG. 3b shows a further exemplary embodiment, wherein the exemplary embodiment substantially represents an extension of the lattice structure 10 according to FIG. 3a. Hereby, a plurality of first wire elements 11 superimpose a single second wire element 12. The second wire element 12 can also superimpose one or a plurality of first wire elements 11 or be braided with the first and/or further second wire elements 11, 12. The plurality of first wire elements 11 can also be superimposed or braided with each other.

FIGS. 4a and 4b show an exemplary embodiment of the medical device according to the invention with a lattice structure 10 formed from two wire elements 11, 12. Hereby, the first wire element 11 has a smaller angle α′ with respect to the rotational axis R than the second wire element 12. In the perspective representation according to FIGS. 4a and 4b, the angle difference between the wire elements 11, 12 may be identified from the different inclination of the spiral wire elements 11, 12. In particular, the angle difference is also evident from the fact that, with a rotation of 360° about the rotational axis, the wire element 11 has a greater axial longitudinal extension along the rotational axis R than a rotation or winding of the second wire element 12. At the axial ends of the lattice structure 10, the wire elements 11, 12 are connected to each other. It is possible for the two wire elements 11, 12 to be formed from a single wire or connected by a force-fit connection to the axial ends of the lattice structure 10 in such a way that the lattice structure 10 is substantially formed from a single continuous wire. In the exemplary embodiment according to FIG. 4a, the wire elements 11, 12 are connected at the axial end of the lattice structure under an obtuse angle so that the wire spirals formed by the wire elements 11, 12 are arranged substantially in opposite directions. On the other hand, according to FIG. 4b, the connection between the wire elements 11, 12 at the axial end of the lattice structure 10 forms an acute angle, i.e. the wire elements 11, 12 are substantially arranged in the same direction along the rotational axis R of the lattice structure 10, wherein the wire spirals have different inclinations. The wire elements 11, 12 can generally be connected by means of a positive connection at the axial ends of the lattice structure 10 or the stabilisation section. The wire elements 11, 12 can form terminating knots. Other types of connection are possible. The wire elements 11, 12 can also be connected in a central section of the lattice structure 10 or fixed to each other.

Furthermore, the lattice structure 10 can be fixed by the braid geometry so that no connection of the wire elements 11, 12 in the axial end sections is required. The frictionally-engaged connection of the superimposed, overlapping or braided wire elements 11, 12 in the intersection regions 13 or knots is sufficient to fix the lattice structure 10. In the case of a lattice structure 10 with a stabilisation section and at least one further section, therefore, a transition region between the stabilisation section and the further section can form a continuous transition. In the transition region, the wire elements 11, 12 can be loosely connected to each other or only connected by frictional engagement. Hereby, the asymmetrical configuration of the lattice structure 10 is retained unchanged.

A detailed view of an asymmetrical braid configuration according to a further exemplary embodiment of the medical device according to the invention is shown in FIG. 5. The braided mesh structure or the lattice structure 10 comprises a plurality of first wire elements 11 and a plurality of second wire elements 12, wherein the first wire elements 11 in the plane of the drawing run diagonally from bottom left to top right and the second wire elements 12 run diagonally from bottom right to top left. In FIG. 5, the rotational axis R (not shown) runs vertically in the plane of the drawing.

The asymmetry, i.e. the different braiding angles of the wire elements 11, 12, is identifiable from the fact that the braided wire elements 11, 12 do not form any rhombic or quadratic cells 14. Instead, the wire elements 11, 12 form rectangular cells 14 with diagonals arranged not perpendicularly but facing each other. Other geometrical designs of the cells 14 are possible.

The wire elements 11, 12 are furthermore braided with each other, wherein each first wire element 11 is guided once under and once over a second wire element 12. Other braid configurations are also possible, for example, two or a plurality of first wire elements 11 can be guided over and below one or a plurality of wire elements 12.

It is evident from FIGS. 2a and 2b that the asymmetrical braid configuration is obtained by the superimposition of at least two inherently symmetrical substructures or braids. In the exemplary embodiment according to FIG. 2, the first structure 15 comprises two first wire elements 11 with the same braiding angle α′ arranged symmetrically to each other. The first substructure 15, therefore, has an inherently symmetrical structure. The second substructure 16 comprises four wire elements 12 each having the same second braiding angle α″. The second substructure 16, therefore, also has an inherently symmetrical design. The respective braiding angles α′, α″ of the first substructure 15 and the second substructure 16 differ from each other. In particular, the first braiding angle α′ of the first substructure 15 is smaller than the second braiding angle α″ of the second substructure 16. The superimposition or overlapping of the two substructures 15, 16 produces the asymmetrical overall structure of the device, which is specifically the result of the fact that the first and second wire elements 11, 12 each have different braiding angles α′ α″.

The same applies to the device according to FIG. 2b, which substantially only differs from the device according to FIG. 2a in the position of the imaginary plane L and the imaginary straight line R′, R″.

It is also possible for at least two different substructures with different braiding angles to be combined with each other or to interact. The invention is not restricted to a combination of two inherently symmetrical substructures, but covers the combination of more than two substructures, for example three, four or more substructures. The individual substructures are each designed inherently symmetrical with the same braiding angle. The braiding angles of the individual structures are different so that, for example, a first, second, third or fourth braiding angle is present. The braiding angle of the further (more than two) structures can also be of an equal size. Different structures can be arranged in series in the axial longitudinal direction of the device and superimpose one or a plurality of structures, wherein the braiding angle of the respectively superimposing structures are different so that the properties the device or of the stent, are variable, for example with respect to the rigidity, radial force, compliance etc.

It is also possible for the medical device, in particular the braided mesh structure 10, partially to comprise an asymmetrical braid. For example, the lattice structure 10 can comprise a stabilisation section arranged in a central region of the lattice structure 10. The marginal regions of the lattice structure 10 can comprise a symmetrical braid. A lattice structure 10 of this kind is, for example, particularly suitable for use as an artificial vessel, i.e. a graft, since the symmetrical section of the lattice structure 10 can extend into the marginal regions and hence become anchored in the vessel, while, on the other hand, sufficient stability is ensured in the central region of the lattice structure 10 even in the case of high pressure fluctuations within the vessel.

Generally, a large angle difference between the wire elements 11, 12 achieves high stability of the lattice structure 10. Hereby, the stability in axial direction effected by a wire element 11, 12 with a relatively planar or small angle α′, α″ to the rotational axis R, while, on the other hand, the radial stability is effected by increasing the angle α′, α″ of a further wire element 11, 12. The combination of two wire elements 11, 12, wherein a first wire element 11 has a smaller or larger angle α′ than a second wire element 12, increases both the axial and the radial stability of the lattice structure 10.

The wire elements 11, 12 or all the wire elements forming the lattice structure comprise the same material in order to achieve the high stability of the lattice structure 10. If the device is used as a stent or artificial vessel or vascular prosthesis, a pseudoelastic material, for example Nitinol, is suitable. The material is conditioned so that, at body temperature, pseudoelastic extension takes place. Despite the asymmetrical braid configuration, the pseudoelastic properties enable a small change in diameter so that, in implanted state, a stent or a stent-graft can exert a radial force on the vessel to fix the implant adequately in the vessel.

### LIST OF REFERENCE NUMBERS

10 Lattice structure

11 First wire element

12 Second wire element

13 Intersection region

14 Cell

15 First structure

16 Second structure

R Rotational axis

R′ First straight line

R″ Second straight line

S′ First intersection point

S″ Second intersection point

α′ First angle

α″ Second angle

U Demarcation line

1-24. (canceled)

25. A medical device comprises a rotation-symmetrical lattice structure (10) having at least first and second wire elements (11, 12) wound about a common rotational axis R in a spiral shape and forming first and second intersection points S′, S″ with a common plane L arranged perpendicular to the rotational axis, wherein a first straight line R′ runs through the first intersection point S′ and a second straight line R″ runs through the second intersection point S″, the straight lines being arranged parallel to the rotational axis R and enclosing an acute angle (α′, α″) with one of the two wire elements (11, 12), and wherein the two angles (α′, α″) are different.

26. The medical device according to claim 25, wherein all the wire elements (11, 12) of the lattice structure (10) comprise a same inelastic material, such that the lattice structure (10) has a substantially rigid geometry.

27. The medical device according to claim 25, wherein all the wire elements (11, 12) of the lattice structure (10) comprise a same elastic material, such that the rotation-symmetrical lattice structure (10) has a substantially variable geometry.

28. The medical device according to claim 25, wherein the angle difference between the first angle (α′) and the second angle (α″) is at least 2°.

29. The medical device according to claim 25, wherein the angle difference between the first angle (α′) and the second angle (α″) is at most 10°.

30. The medical device according to claim 25, wherein the first angle (α′) is smaller than 45°, and the second angle (α″) is greater than 2°.

31. The medical device according to claim 25, wherein a ratio between a number of the first wire elements (11) and a number of the second wire elements (12) is at most 1:1.

32. The medical device according to claim 25, wherein the first wire element (11) with the first straight line R′ encloses a smaller angle α′ than the second wire element (12) with the second straight line R″.

33. The medical device according to claim 25, wherein the lattice structure has at least one stabilization section, which extends at least partially in an axial direction along the lattice structure (10), and wherein the first angle (α′) along the stabilization section is always greater or always smaller than the second angle (α″).

34. The medical device according to claim 33, wherein the stabilization section forms an axially central section (16) and/or axial end section (17) of the lattice structure (10).

35. The medical device according to claim 33, wherein the lattice structure has two stabilization sections each forming an axial end section (17) of the lattice structure (10).

36. The medical device according to claim 25, wherein the first angle (α′) and/or the second angle (α″) varies at least in sections along the lattice structure (10), optionally along a straight line R′, R″ running parallel to the rotational axis R.

37. The medical device according to claim 25, wherein the rotation-symmetrical lattice structure (10) is embodied substantially as a tube, which is stent-like.

38. The medical device according to claim 25, wherein the at least first and second wire elements (11, 12) along the lattice structure (10) are wound in a coil manner at least in sections.

39. The medical device according to claim 25, wherein the at least first and second wire elements (11, 12) along the lattice structure (10) are braided with each other at least in sections.

40. The medical device according to claim 25, wherein the at least first and second wire elements (11, 12) are connected to each other at least at an axial end or in an intersection region (13) of the lattice structure (10), the connection being selected from positive, non-positive, force-fit, glued, and welded connections.

41. The medical device according to claim 40, wherein the intersection region (13) abuts a stabilization section.

42. The medical device according to claim 27, wherein the wire elements (11, 12) comprise a material selected from a shape-memory material and a pseudoelastic material, optionally a nickel-titanium alloy.

43. The medical device according to claim 27, wherein the wire elements (11, 12) comprise a plastic selected from polyester, polyamide, polypropylene, and polyethylene, optionally HDPE or UHMWPE.

44. The medical device according to claim 25, wherein the lattice structure comprises a flexible enclosure, extending, at least in sections, in at least one of a circumferential direction and an axial direction along the lattice structure (10).

45. The medical device according to claim 44, wherein the flexible enclosure comprises a plastic, optionally polyurethane, silicone or PTFE.

46. The medical device according to claim 25, wherein a plurality of the first wire elements (11) have the first angle α′ and together form a first symmetrical structure (15) and a plurality of the second wire elements (12) have the second angle α″ and together form a second symmetrical structure (16), and wherein the first structure (15) and second structure (16) are superimposed, such that the first and second wire elements (11, 12) with different first and second angles α′, α″ are assigned to each other.

47. A method for producing a medical device according to claim 25, the method comprising winding at least two wire elements (11, 12) about a common rotational axis in a spiral shape in such a way that the wire elements (11, 12) each enclose different acute angles (α′, α″) with a straight line R′, R″ running parallel to the rotational axis Rat least in one plane L arranged perpendicular to the rotational axis R.

48. The method according to claim 47, wherein the wire elements (11, 12) are braided and/or wound with a textile machine, optionally a spinning machine or weaving machine.
